(19) United States
(12) Patent Application Publication
(10) Pub. No.: US 2010/0215594 A1
(43) Pub. Date: Aug. 26, 2010

(54) Tyrosinase Inhibitors

(75) Inventors: Sylvia Huber, Heppenheim (DE); Teresa Mujica-Fernaud, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK PATENT GMBH, Darmstadt (DE)

(21) Appl. No.: 12/673,312

(22) PCT Filed: Jul. 17, 2008
(86) PCT No.: PCT/EP2008/005830

§ 371 Date: Feb. 12, 2010

(30) Foreign Application Priority Data
Aug. 13, 2007 (DE) 10 2007 038 097.8

(51) Int. Cl. A61K 8/49; C07D 207/28; A61K 31/4015; C07C 229/12; A61K 31/222; A61Q 17/04; A61P 17/00

(52) U.S. Cl. 424/59; 548/534; 514/423; 560/173; 514/551

(57) ABSTRACT

The invention relates to compounds of the formula I where the substituents AA₁, AA₂, o and R have a meaning indicated in claim 1, and to the salts and solvates and mixtures thereof, to a process for the preparation thereof, preparations and the use thereof.

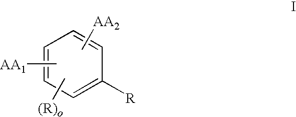

The invention relates to compounds of the formula I

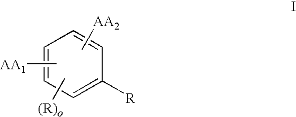

where the substituents AA₁, AA₂ and R have a meaning indicated in Claim 1, and to salts and solvates and mixtures thereof, to a process for the preparation thereof, to compositions and to the use thereof, in particular as tyrosinase inhibitors, suitable for lightening human skin or for the prophylaxis and/or treatment of pigment defects, such as hyperpigmentation, freckles, age spots, sun spots and environmentally induced skin ageing.

Skin and hair colour are dependent on the content, size and type of melanin (a nitrogen-containing dark dye) which is produced from melanocytes, the cells which are capable of melanin formation. Starting from tyrosine and with the aid of various melanocyte-specific enzymes, such as tyrosinase or tyrosinase-related proteins, melanin is produced within the melanosomes, with subsequent conversion of the melanosomes into keratinocytes.

Although the melanin in the skin is suitable protection against UV radiation, darker or overpigmented skin can, as already mentioned, affect beauty and result in serious aesthetic problems. Hyperpigmented skin conditions or lesions contain melasma (also known as chloasma), i.e. yellowish-brown spots of irregular shape.

In the case of pigment spots, a distinction is generally made between freckles (ephelides), age spots (lentigines), so-called age warts (verrucae seborrhoica) and hyperpigmentation (for example chloasma or melasma) and the sun very frequently plays an important role here. People with very pale skin and reddish hair tends towards freckles. By contrast, hyperpigmentation (chloasma) is frequently found in women who regularly introduce oestrogens into their body.

Prevention can be achieved, in particular, by regular sun protection with a high light protection factor. However, once it has arisen, various possibilities, such as lasers, dermabrasion or other electrosurgical methods and so-called bleaching creams, are available for removing the unattractive age spots. The latter alternative (bleaching creams) have the advantage that they are significantly less expensive for the patient than the electrosurgical methods.

A large number of compounds having a skin-lightening action is available on the market for the treatment of pigment spots.

These are, inter alia, compounds such as kojic acid, arbutin, aloesin or rucinol, which suppress melanin production in the skin. They delay the conversion of tyrosine into melanin by blocking the enzyme tyrosinase.

However, these compounds have a number of disadvantages, such as, for example, low depigmentation efficiency, side effects, such as skin irritation or skin exfoliation (skin peeling), cell damage, low penetration through the skin or low stability of the formulations. There is therefore a need for novel skin tighteners having higher effectiveness.

The object of the present invention was therefore to prepare novel cornpounds which have the ability to lighten skin.

Unsubstituted 4-alkylresorcinols are compounds which are known to have the ability to reduce skin pigmentation. This is described in many publications, for example in EP 0341664, EP 904774, WO 2004/103940, EP 1317425, US 2006/0210498, US 2004/0109832.

A process for the preparation of 4-alkylresorcinol mono- and diacetate is described in WO 2004/052827. U.S. Pat. No. 6,863,897 and U.S. Pat. No. 6,869 disclose cosmetic compositions comprising 4-alkylresorcinol or 4-alkylresorcinol diacetate.

Surprisingly, it has now been found that compounds of the formula I, as described below, have excellent skin-lightening properties. They suppress the synthesis of melanin, prevent melanin overproduction and are thus suitable for the treatment of pigment spots of all types.

The present invention therefore relates firstly to compounds of the formula I

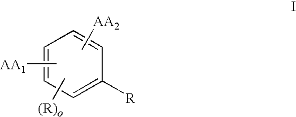

where
AA₁ and AA₂ each, independently of one another, denote OH or a radical of a proteinogenic or non-proteinogenic amino acid,
R in each case, independently of one another, optionally substituted,
denotes a linear or branched alkyl group having 1 to 12 C atoms, cycloalkyl having 3 to 9 C atoms or cycloalkenyl having 5 to 9 C atoms,
o is 0, 1 or 2,
with the proviso that AA₁ and AA₂ are not simultaneously OH, and salts and solvates thereof.

Solvates of the compounds of the formula I are taken to mean adductions of inert solvent molecules onto the compounds of the formula I, which form owing to their mutual attractive force. Solvates are, for example, mono- or dihydrates or addition compounds with alcohols, such as, for example, with methanol or ethanol.

If the compounds of the formula I have at least one chiral centre, they can occur in a number of stereoisomeric forms. All these forms (for example D and L forms) and mixtures thereof (for example the DL forms) are included in the formula.

Proteinogenic amino acids are, for example, glycine, alanine, serine, cysteine, phenylalanine, tyrosine, threonine, methionine, valine, leucine, isoleucine, arginine, lysine, aspartic acid, asparagine, glutamic acid, glutamine, proline, tryptophan or histidine. Non-proteinogenic amino acids are, for example, β-alanine, γ-aminobutyric acid, 2-aminoisobutyric acid, N,N′-dimethyl-4-aminobutyric acid, N,N′-dimethylglycine, betaines, creatine, carnitine, sarcosine, betanine, 2-pyrrolidinone-5-carboxylic acid, ectoin, hydroxyectoin, thyroxine, diiodotyrosine, L-dopa, phosphinothricine, 1-aminocyclopropanecarboxylic acid or niacin.

In a preferred embodiment, the radical R conforms to one of the sub-formulae II, III or IV:

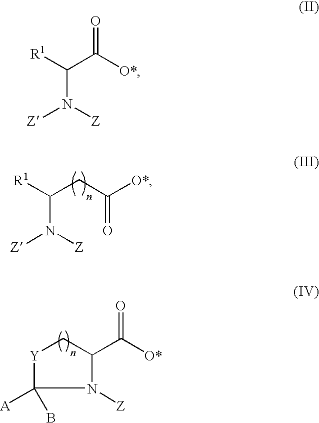

where
Z and Z′ each independently denote H, R, OCOCH₃, OCOC₆H₅, CH₂C₆H₅ or OCOC(CH₃)₃,
R¹ denotes H, or a linear or branched alkyl group having 1 to 30 C atoms, optionally substituted by OH, OR, SH, SR, NH₂, NHR, NR₂, substituted or unsubstituted guanidinium, (NR₃)⁺, COOH, COOR, CONH₂, CONHR, CONR₂, COR, Ar or Het,
R in each case, independently of one another, optionally substituted, denotes a linear or branched alkyl group having 1 to 12 C atoms, cycloalkyl having 3 to 9 C atoms or cycloalkenyl having 5-9 C atoms,
Y denotes —CH₂— or —NZ—,
A and B each, independently of one another, denote H, R or OH, or A and B, together with the C atom to which they are linked, may form a carbonyl group,
n is 1, 2 or 3,
Ar denotes phenyl which is unsubstituted or mono-, di- or trisubstituted by R, OH or OR,
Het denotes a saturated or unsaturated mono- or bicyclic heterocyclic radical having 5 to 13 ring members, where 1, 2 or 3 N and/or 1 or 2 S or O atoms may be present, and the heterocyclic radical may be mono- or poly-substituted by R, OH or OR, and
double bonds may also be present in the ring in formula IV.

A preferred group of compounds of the formula I comprises compounds of the formula Ia

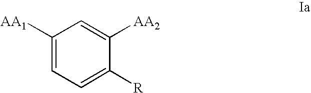

where the substituents AA₁, AA₂ and R have one of the meanings described in greater detail above or below.

In the formulae I and Ia, R denotes a linear or branched alkyl group having 1 to 12 C atoms, preferably methyl, furthermore ethyl, n-propyl, isopropyl, n-butyl, sec-butyl or tert-butyl, furthermore also pentyl, 1-, 2- or 3-methyl-butyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethyl-butyl, 1-ethyl-1-methylpropyl, 1-ethyl-2-methylpropyl, 1,1,2- or 1,2,2-trimethylpropyl, or cycloalkyl having 3 to 9 C atoms, preferably cyclopentyl, cyclohexyl or cycloheptyl, or cycloalkenyl having 5 to 9 C atoms, preferably cyclopentenyl, cyclohexenyl or cycloheptenyl.

R may optionally also be substituted, i.e. at least one H may be substituted, for example, by a substituent selected from the group linear, branched or cyclic alkyl having 1 to 12 C atoms, linear, branched or cyclic alkenyl having 1 to 12 C atoms.

R is preferably a linear or branched alkyl group having 1-12 C atoms. R particularly preferably stands for hexyl.

Preferred compounds of the formula I are compounds in which o=0 or 1, particularly preferably those in which o=0.

In the sub-formulae II and III, R¹ denotes H or a linear or branched, optionally substituted alkyl group having 1 to 30 C atoms.

R in the definition of R¹, Z, Z′, A, B, Ar and Het preferably denotes methyl, ethyl or propyl, particularly preferably methyl.

Ar denotes phenyl which is unsubstituted or mono-, di- or trisubstituted by R, OH or OR, for example phenyl, o-, m- or p-methylphenyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-methoxyphenyl, o-, m- or p-ethoxyphenyl or o-, m- or p-propoxyphenyl.

The heterocyclic radical having 5 to 13 ring members, where 1, 2 or 3 N and/or 1 or 2 S or O atoms may be present, preferably denotes substituted or unsubstituted 2- or 3-furyl, 2- or 3-thienyl, 1-, 2- or 3-pyrrolyl, 1-, 2-, 4- or 5-imidazolyl, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -4- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 2-, 3-, 4-, 5- or 6-2H-thiopyranyl, 2-, 3- or 4-4H-thiopyranyl, 3- or 4-pyridazinyl, pyrazinyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-1H-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 1-, 2-, 3-, 4-, 5-, 6-, 7- or 8-quinolinyl, 1-, 3-, 4-, 5-, 6-, 7- or 8-isoquinolinyl, 1-, 2-, 3-, 4- or 9-carbazolyl, 1-, 2-, 3-, 4-, 5-, 6-, 7-, 8- or 9-acridinyl, 3-, 4-, 5-, 6-, 7- or 8-cinnolinyl, 2-, 4-, 5-, 6-, 7- or 8-quinazolinyl or 1-, 2- or 3-pyrrolidinyl.

Het is particularly preferably 1-, 2-, 4- or 5-imidazolyl or 1-, 2-, 3-, 4-, 5-, 6- or 7-1H-indolyl.

The sub-formula IV preferably does not have a double bond in the ring.

A preferred group of compounds comprises compounds of the formula Ia where AA₁ conforms to the sub-formula II and AA₂ denotes OH, or AA₂ conforms to the sub-formula II and AA₁ denotes OH.

A particularly preferred group of compounds comprises compounds of the formula Ia where AA₁ conforms to the sub-formula III and AA₂ denotes OH, or AA₂ conforms to the sub-formula III and AA₁ denotes OH.

A particularly preferred group of compounds comprises compounds of the formula Ia where AA₁ conforms to the sub-formula IV and AA₂ denotes OH, or AA₂ conforms to the sub-formula IV and AA₁ denotes OH.

Further preferred combinations are disclosed in the Claims.

Particularly preferred compounds of the formula I or la are
4-hexyl-3-hydroxyphenyl 2-pyrrolidinone-5-carboxylate,
2-hexyl-5-hydroxyphenyl 2-pyrrolidinone-5-carboxylate,
4-hexyl-3-hydroxyphenyl 4-dimethylaminobutyrate,
2-hexyl-5-hydroxyphenyl 4-dimethylaminobutyrate,
4-hexyl-3-hydroxyphenyl dimethylaminoacetate,
2-hexyl-5-hydroxyphenyl dimethylaminoacetate,
   and salts and solvates thereof.

The compounds of the formula I according to Claim 1 and also the starting materials for the preparation thereof are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants known per se which are not mentioned here in greater detail.

The invention furthermore also relates to a process for the preparation of compounds of the formula I or Ia and salts and solvates thereof, characterised in that

(a) a compound of the formula V or Va

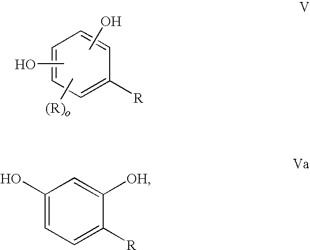

in which R and a have a meaning indicated in Claim 1,
is reacted with a proteinogenic or non-proteinogenic amino acid or with a compound of the formula VI

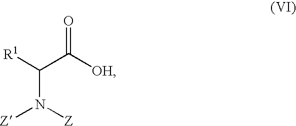

of the formula VII

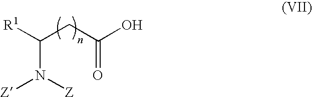

or
of the formula VIII

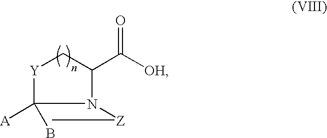

where the substituents Z, Z′, R¹, Y, A, B and n have one of the meanings indicated above, and
(b) optionally a basic or acidic compound of the formula I is converted into one of its salts or solvates by treatment with an acid or base.

The compounds of the formulae V and Va are known compounds which are prepared by various methods or in some cases are also commercially available.

For example, resorcinol or a hydroxyphenol in general can be reacted with a carboxylic acid RCOOH in the presence of zinc chloride, and the resultant condensate can be reduced using zinc/amalgam/hydrochloric acid, analogously to Lille. J. Bitter et al, Inst. Slantsev 1969, 18, 127.

Furthermore, resorcinol or a hydroxyphenol in general can be reacted with an alcohol HO—CH₂—R in the presence of an aluminium catalyst at high temperatures of 200 to 400° C., analogously to GB 1581428.

Further methods for the preparation of the compounds of the formula Va are also described in EP 0341664, EP 904774, WO 2004/103940, EP 1317425, US 2006/0210498, US 2004/0109832.

The complete disclosure content of all applications, patents and publications mentioned above and below is incorporated into this application by way of reference.

Proteinogenic and non-proteinogenic amino acids are generally commercially available or can be isolated from natural sources. The compounds of the formulae VI, VII and VIII are generally commercially available. They can furthermore also be prepared synthetically, as described in the literature in numerous standard works of chemistry.

For example, the compounds of the formulae VI, VII and VIII can be obtained by the so-called Strecker synthesis (A. Strecker, Justus Liebigs Ann. Chem. 1850, 75, 27-45) and/or variants thereof (inter alia J. Mulzer et al, Organic Synthesis Highlights, VCH, Weinheim, 1991, 303; H. Kunz et al., Angew. Chem. 1987, 99, 595; D. Enders et al, Tetrahedron Lett. 2003, 44, 8479).

The compounds of the formulae I and Ia can easily be prepared in a one-step synthesis from the compounds of the formulae V and Va by esterification using the acids of the formula VI, VII or VIII. A wide variety of methods are disclosed in this respect in the literature.

The coupling reaction is preferably carried out in the presence of an activating reagent, for example a carbodiimide, such as dicyclohexylcarbodiimide (DCC), N-(3-dimethylaminopropyl)-N′-ethylcarbodiimide hydrochloride (EDC), diisopropylcarbodiimide (DIC), or with 4-dimethylaminopyridine (DMAP) as acylation catalyst (Aldrichchimia Acta, 36, 1, 2003) in an inert solvent, for example a halogenated hydrocarbon, such as dichloromethane, an ether, such as tetrahydrofuran or dioxane, an amide, such as DMF or dimethylacetamide, a nitrile, such as acetonitrile, in dimethyl sulfoxide or in the presence of these solvents, at temperatures between about −10 and 40°, preferably between 0 and 30°. Depending on the conditions used, the reaction time is between a few minutes and a number of days.

The addition of the coupling reagent TBTU (O-(benzotriazol-1-yl)-N,N,N′,N′-bis(tetramethylene)uronium tetrafluoroborate) or O-(benzotriazol-1-yl)-N,N,N′,N′-bis(tetramethylene)uronium hexafluorophosphate has proven particularly advantageous, since, in the presence of one of these compounds, only slight racemisation occurs and no cytotoxic by-products are formed.

The compounds of the formula I or Ia can also be formed if an alcohol of the formula V or Va and a carboxylic acid of the formula VI, VII or VIII are combined with an acid as catalyst (such as, for example, concentrated sulfuric acid).

There are a number of further ways of converting alcohols and acids or acid derivatives into esters, for example the reaction between an alcohol and an acid chloride (Schotten-Baumann method), an alcohol and an acid anhydride, the Steglich esterification or the Mitsunobu reaction.

A compound of the formula I can be converted into the associated acid-addition salt using an acid, for example by reaction of equivalent amounts of the base and acid in an inert solvent, such as ethanol, followed by evaporation. Particularly suitable acids for this reaction are those which give physiologically acceptable salts. Thus, it is possible to use inorganic acids, for example sulfuric acid, sulfurous acid, dithionic acid, nitric acid, hydrohalic acids, such as hydrochloric acid or hydrobromic acid, phosphoric acids, such as, for example, orthophosphoric acid, sulfamic acid, furthermore organic acids, in particular aliphatic, alicyclic, araliphatic, aromatic or heterocyclic mono- or polybasic carboxylic, sulfonic or sulfuric acids, for example formic acid, acetic acid, propionic acid, hexanoic acid, octanoic acid, decanoic acid, hexadecanoic acid, octadecanoic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, benzenesulfonic acid, trimethoxybenzoic acid, adamantanecarboxylic acid, p-toluenesulfonic acid, glycolic acid, embonic acid, chlorophenoxy-acetic acid, aspartic acid, glutamic acid, proline, glyoxylic acid, palmitic acid, para-chlorophenoxyisobutyric acid, cyclohexanecarboxylic acid, glucose 1-phosphate, naphthalenemono- and -disulfonic acids or laurylsulfuric acid. Salts with physiologically unacceptable acids, for example picrates, can be used for the isolation and/or purification of the compounds of the formula I. On the other hand, compounds of the formula I can be converted into the corresponding metal salts, in particular alkali metal or alkaline-earth metal salts or into the corresponding ammonium salts using bases (for example sodium or potassium hydroxide or carbonate).

In general, it should be noted in the one-pot synthesis described that a mixture of regioisomeric compounds generally arises, which, however, can then optionally be separated into the individual compounds. Standard separation and work-up methods are distillation, recrystallisation and chromatography, for example liquid chromatography and the associated methods, such as thin-layer chromatography and column chromatography (low pressure chromatography, flash chromatography and high-performance chromatography, also known as HPLC).

The invention therefore furthermore also relates to a mixture comprising at least one compound of the formula Ia-1

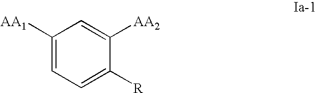

and at least one compound of the formula Ia-2

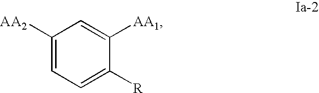

where the substituents AA₁, AA₂ and R are identical and have one of the meanings indicated in Claim 1 or 2 or the meanings preferably mentioned.

The invention furthermore also relates to a mixture comprising at least one compound of the formula Ia-1 and/or at least one compound of the formula Ia-2 and at least one compound of the formula Va

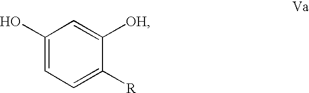

where the substituents AA₁, AA₂ and R are identical and have one of the meanings indicated in Claim 1 or 2 or the meanings preferably mentioned.

Advantages of the compounds according to the invention or of the mixture according to the invention as skin-lightening active compounds or of compounds preferably indicated or mixtures thereof may be, in particular:
they can easily be incorporated into compositions,
they have increased stability in compositions,
they do not exhibit skin irritation,
they exhibit high and long-lasting activity with respect to their action as skin-lightening active compounds.

On the one hand, the compounds and/or mixtures according to the invention are tyrosinase inhibitors, as confirmed in the example part, and, owing to this property, exhibit the desired activity as skin lighteners. On the other hand, after application of the compounds and/or mixtures according to the invention, preferably in compositions, to the skin, it is possible for enzymes in the skin, for example lipases or esterases, to break the ester bond present. The compounds of the formula V or Va thus formed in vivo are known skin lighteners and develop their action at least additively, or even synergistically. The proteinogenic or non-proteinogenic amino acids formed in this reaction or the compounds of the formula VI, VII or VIII have further properties which are advantageous for the skin. For example, amino acids regulate the moisture content of the skin, as described in A. V. Rawlings & C. R. Harding, Dermatologic Therapy, 2004, 17, 43. It has also been reported that amino acids can even reduce in vitro pigmentation in melanocytes (M. Ishikawa et al, Bio. Pharm. Bull. 2007, 30(4), 677-681).

The present invention furthermore relates to a composition or preparation comprising at least one compound of the formula I or Ia, as described above, or a mixture at least of the compounds of the formula Ia-1 or Ia-2, with the embodiments as described above, and at least one vehicle which is suitable for topical applications.

Suitable for topical purposes means suitable for a form which can be applied locally, in particular to the surface.

The compositions are usually either compositions for topical application, for example cosmetic, pharmaceutical or dermatological formulations, or foods or food supplements. In this case, the compositions comprise a cosmetically, pharmaceutically or dermatologically suitable vehicle and, depending on the desired property profile, optionally further suitable ingredients. The topical compositions are preferably employed as cosmetic or dermatological composition, particularly preferably as cosmetic composition. In the case of food supplements, a vehicle which is suitable for foods is used.

For the purposes of the present invention, the term agent, preparation or formulation is used synonymously alongside the term composition.

As described above, the compounds of the formula I or Ia or a mixture are or is typically employed in accordance with the invention in amounts of 0.01 to 20% by weight, preferably in amounts of 0.05% by weight to 10% by weight. The person skilled in the art is presented with absolutely no difficulties here in selecting the amounts correspondingly depending on the intended action of the composition.

It is furthermore advisable, in particular for use as skin-lightening composition or as cosmetic and/or pharmaceutical composition for the prophylaxis and/or treatment of pigment defects, such as hyperpigmentation, freckles, age spots, sun spots and environmentally induced skin ageing, for the compositions according to the invention to comprise one or more antioxidants and/or one or more vitamins.

The use of antioxidants generally enables a protective action to be achieved against oxidative stress or against the action of free radicals, where the person skilled in the art is presented with absolutely no difficulties in selecting antioxidants which act suitably quickly or in a time-delayed manner.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothio-glucose, propylthiouracil and other thiols (for example thioredoxin, gluta-thione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homo-cysteine sulfoximine, buthionine sulfones, penta-, hexa- and heptathionine sulfoximine) in very low tolerated doses (for example pmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), and coniferyl benzoate of benzoin resin, rutinic acid and derivatives thereof, α-glycosyl rutin, ferulic acid, fur-furylideneglucitol, carnosine, butylhydroxytoluene, butylhydroxyanisoie, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also described in WO 2006/111233 and WO 2006/111234.

Suitable antioxidants are also compounds of the general formula A or B

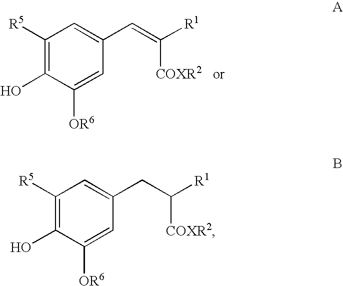

in which
R¹ can be selected from the group —C(O)CH₃, —CO₂R³, —C(O)NH₂ and —C(O)N(R⁴)₂,
X denotes O or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes linear or branched alkyl having 1 to 8 C atoms or linear or branched alkoxy having 1 to 8 C atoms, and
R⁶ denotes linear or branched alkyl having 1 to 8 C atoms, preferably derivatives of 2-(4-hydroxy-3,5-dimethoxybenzylidene)malonic acid and/or 2-(4-hydroxy-3,5-dimethoxybenzyl)malonic acid, particularly preferably bis-(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-di-methoxybenzyl)malonate (for example RonaCare® AP).

Mixtures of antioxidants are likewise suitable for use in the compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed with compounds of the formula I or Ia in such preparations in percent by weight ratios in the range from 1000:1 to 1:1000, preferably in percent by weight ratios of 100:1 to 1:100.

The compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinal, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin C and derivatives thereof, DL-α-tocopherol, tocopherol E acetate, nicotinic acid, pantothenic acid and biotin. Vitamins are usually employed here with compounds of the formula I or Ia in percent by weight ratios in the range from 1000:1 to 1:1000, preferably in percent by weight ratios of 100:1 to 1:100.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F., Soffers, I. M. C. M. Rietjens; Free Radical Biology & Medicine 2001, 31(7), 869-881, are investigating the pH dependence of the antioxidant action of hydroxyflavones. Quercetin exhibits the greatest activity amongst the structures investigated over the entire pH range.

In order that the compounds of the formula I or Ia are able to develop their positive action on the skin particularly well, it may be preferred to allow the compounds of the formula I or Ia to penetrate into deeper skin layers. Several possibilities are available for this purpose. Firstly, the compounds of the formula I or Ia can have an adequate lipophilicity in order to be able to penetrate through the outer skin layer into epidermal layers. As a further possibility, corresponding transport agents, for example liposomes, which enable transport of the compounds of the formula I or Ia through the outer skin layers may also be provided in the composition. Finally, systemic transport of the compounds of the formula I or Ia is also conceivable. The composition is then designed, for example, in such a way that it is suitable for oral administration.

It is also advantageous to administer the compounds of the formula I or Ia in encapsulated form, for example as cellulose or chitin capsules, in gelatine or wax matrices or encapsulated with cyclodextrins.

Compositions which are preferred in accordance with the invention also comprise UV filters besides the at least one compound of the formula I or Ia or a mixture of at least one compound of the formula Ia-1 and a compound of the formula Ia-2, optionally also comprising at least one compound of the formula Va.

In principle, all UV filters are suitable for combination with the compounds of the formula I or Ia according to the invention. Particular preference is given to UV filters whose physiological acceptability has already been demonstrated. Both for UVA and UVB filters, there are many proven substances which are known from the specialist literature, for example

benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyl-dibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for example Eusolex® 2292), isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethyl-cyclohexyl salicylate (for example Eusotex® HMS),
4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25),
phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan®AP) or 2,2-(1,4-phenylene)bisbenzimidazole-6-sulfonic acid,
and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

These organic UV filters are generally incorporated into cosmetic formulations in an amount of 0.5 to 10 percent by weight, preferably 1-8% by weight.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (INCI: Drometrizole Trisiloxane, for example Mexoryl® XL),
α-(trimethylsilyl)-ω-[trimethylsilyl)oxy]poly[oxy(dimethyl [and approximately 6% of methyl[2-[p-[2,2-bis(ethoxycarbonyl]vinyl]phenoxy]-1-methylenethyl] and approximately 1.5% of methyl[3-[p-[2,2-bis(ethoxy-carbonyl)vinyl)phenoxy)propenyl) and 0.1 to 0.4% of (methylhydrogen]-silylene]] (n≈60) (CAS No. 207 574-74-1) (INCI: Polysilicone-15, for example Parsol® SLX),
2,2′-methylenebis(6-(2H-benzotriazol-2-yl)-4-(1,1,3,3-tetramethylbutyl)-phenol) (CAS No. 103 597-45-1) (INCI: Methylene Bis-Benzotriazolyl Tetramethylbutylphenol, for example Tinosorb® M),
2,2′-(1,4-phenylene)bis(1H-benzimidazole-4,6-disulfonic acid, mono-sodium salt) (CAS No. 180 898-37-7),
2,4-bis{[4-(2-ethylhexyloxy)-2-hydroxy]phenyl}-6-(4-methoxyphenyl)-1,3,5-triazine (CAS No. 103 597-45-, 187 393-00-6) (INCI: Bis-Ethyl-hexyloxyphenol Methoxyphenyl Triazine, for example Tinosorb® S) or
2-ethylhexyl 4,4′-[(6-[4-((1,1-dimethylethyl)aminocarbonyl)phenylamino]-1,3,5-triazine-2,4-diyl)diimino]bis(benzoate) (INCI: Diethylhexyl Butamido Triazone, for example Uvasorb® HEB).

Organic UV filters are generally incorporated into cosmetic formulations in an amount of 0.5 to 20 percent by weight, preferably 1-15% by weight.

Conceivable inorganic UV filters are those from the group of titanium dioxides, such as, for example, coated titanium dioxide (for example Eusolex® T-2000, Eusolex® T-AQUA, Eusolex® T-AVO), zinc oxides (for example Sachtotec®), iron oxides or also cerium oxides. These inorganic UV filters are generally incorporated into cosmetic compositions in an amount of 0.5 to 20 percent by weight, preferably 2-10% by weight.

Through combination of one or more compounds of the formula I or Ia with further UV filters, the protective action against harmful effects of UV radiation can be optimised. This gives rise to broad-band protection systems, which can also be supplemented by addition of inorganic UV filters.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin is repeatedly being discussed in the literature. The encapsulation of the corresponding substances which is proposed here suppresses this effect.
In general, encapsulation of individual UV filters or other ingredients enables composition problems caused by the interaction of individual composition constituents with one another, such as crystallisation processes, precipitation and agglomerate formation, to be avoided since the interaction is suppressed.

It is therefore preferred for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active compound (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules are preferably present in compositions according to the invention in amounts which ensure that the encapsulated UV filters are present in the composition in the above-indicated amounts.

The compositions according to the invention may also comprise one or more further skin-lightening active compounds. Skin-lightening active compounds can in principle be all active compounds known to the person skilled in the art. Examples of compounds having skin-lightening activity are hydroquinone, kojic acid, arbutin, aloesin and rucinol.

The compositions according to the invention may in addition comprise further anti-ageing active compounds, anticellulite active compounds or conventional skin-protecting or skin-care active compounds. Skin-protecting or skin-care active compounds can in principle be all active compounds known to the person skilled in the art.

Particularly preferred anti-ageing active compounds are pyrimidinecarboxylic acids, aryl oximes, bioflavonoids, bioflavonoid-containing extracts, chromones and retinoids.

Pyrimidinecarboxylic acids occur in halophilic microorganisms and play a role in osmoregulation of these organisms (E. A. Galinski et al., Eur. J. Biochem., 149 (1985) pages 135-139). Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoin and ectoin derivatives, such as hydroxyectoin, can advantageously be used in medicaments. In particular, hydroxyectoin can be employed for the preparation of a medicament for the treatment of skin diseases. Other areas of application of hydroxyectoin and other ectoin derivatives are typically in areas in which, for example, trehalose is used as additive. Thus, ectoin derivatives, such as hydroxyectoin, can be used as protectant in dried yeast and bacteria cells. Pharmaceutical products, such as non-glycosylated, pharmaceutically active peptides and proteins, for example t-PA, can also be protected with ectoin or its derivatives.

Of the cosmetic applications, particular mention should be made of the use of ectoin and ectoin derivatives for the care of aged, dry or irritated skin. Thus, European patent application EP-A-0 671 161 describes, in particular, that ectoin and hydroxyectoin are employed in cosmetic compositions, such as powders, soaps, surfactant-containing cleansing products, lipsticks, rouge, make-ups, care creams and sunscreen preparations.

Preference is given here to the use of a pyrimidinecarboxylic acid of the following formula:

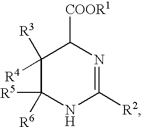

in which R¹ is a radical H or C₁₋₈-alkyl, R² is a radical H or C₁₋₄-alkyl, and R³, R⁴, R⁵ and R⁶ are each, independently of one another, a radical from the group H, OH, NH₂ and C₁₋₄-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid). The compositions according to the invention preferably comprise pyrimidinecarboxylic acids of this type in amounts of up to 15% by weight. The pyrimidinecarboxylic acids are preferably employed here in percent by weight ratios of 100:1 to 1:100 with respect to the compounds of the formula I, with percent by weight ratios in the range 1:10 to 10:1 being particularly preferred.

Of the aryl oximes, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. The compositions here preferably comprise 0.01 to 10% by weight of the aryl oxime, it being particularly preferred for the composition to comprise 0.05 to 5% by weight of aryl oxime.

Known bioflavonoids are, for example, troxerutin, tiliroside, α-glucosylrutin, rutin or isoquercetin, where the said choice is not intended to have a restrictive effect.

Bioflavonoid-containing extracts are, for example, gingko biloba or emblica.

Known anti-ageing substances are also chromones, as described, for example, in EP 1508327, and retinoids, for example retinol (vitamin A), retinoic acid, retinaldehyde and also synthetically modified compounds of vitamin A.

The chromones and retinoids described are simultaneously also effective anticeliulite active compounds. A likewise known anticellulite active compound is caffeine.

The compositions may include or comprise, essentially consist of or consist of the said necessary or optional constituents or ingredients. All compounds or components which can be used in the compositions are either known and commercially available or can be synthesised by known processes.

The one or more compounds of the formula I or Ia or the mixture, as described above, can be incorporated into cosmetic or dermatological cornpositions in the customary manner. Suitable compositions are those for external use, for example in the form of a cream, lotion or gel, or as a solution which can be sprayed onto the skin.

Suitable for internal use are administration forms such as capsules, dragees, powders, tablet solutions or solutions.

Use forms of the compositions according to the invention that may be mentioned are, for example: solutions, suspensions, emulsions, PIT emulsions, pastes, ointments, gels, creams, lotions, powders, soaps, surfactant-containing cleansing preparations, oils, aerosols and sprays. Examples of other use forms are sticks, shampoos and shower compositions. Any desired customary vehicles, assistants and, if desired, further active compounds may be added to the composition.

Preferred assistants originate from the group of the preservatives, stabilisers, solubilisers, colorants, i.e. pigments or dyes, or odour improvers.

Ointments, pastes, creams and gels may comprise the customary vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic use forms are also powder, emulsion and wax make-up, and sunscreen, pre-sun and after-sun preparations.

The preferred composition forms according to the invention include, in particular, emulsions.

Emulsions according to the invention are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of the esters of aromatic carboxylic acid and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group of the branched and unbranched hydrocarbons and hydrocarbon waxes, silicone oils, dialkyl ethers, or the group of the saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18, C atoms. The fatty acid triglycerides may advantageously be selected, for example, from the group of the synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride, dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane, poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, dials or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty substances, as well as water and an emulsifier, as usually used for a formulation of this type.

In a preferred embodiment, the compositions according to the invention comprise hydrophilic surfactants.

The hydrophilic surfactants are preferably selected from the group of the alkylglucosides, acyl lactylates, betaines and coconut amphoacetates.

The alkylglucosides are themselves advantageously selected from the group of the alkylglucosides which are distinguished by the structural formula

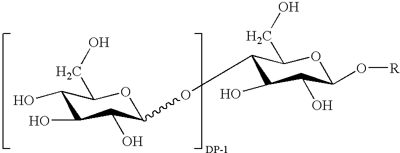

where R represents a branched or unbranched alkyl radical having 4 to 24 carbon atoms and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + ... = \sum \frac{{p}_{i}}{100} \cdot i$$

in which p₁, p₂, p₃ . . . pᵢ represent the proportion of mono-, di-, tri- . . . i-fold glucosylated products in percent by weight. Products which are advantageous in accordance with the invention are those having degrees of glucosylation of 1-2, particularly advantageously of 1.1 to 1.5, very particularly advantageously of 1.2-1.4, in particular of 1.3.

The value DP takes into account the fact that alkylglucosides are generally, as a consequence of their preparation, in the form of mixtures of mono- and oligoglucosides. A relatively high content of monoglucosides, typically in the order of 40-70% by weight, is advantageous in accordance with the invention.

Alkylglucosides which are particularly advantageously used for the purposes of the invention are selected from the group octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and assistants or mixtures which are distinguished by an effective content of the active compounds used in accordance with the invention, for example Plantaren® 1200 (Henkel KGaA), Oramix® NS 10 (Seppic).

The acyllactylates are themselves advantageously selected from the group of the substances which are distinguished by the structural formula

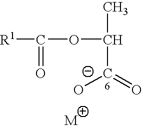

where R¹ denotes a branched or unbranched alkyl radical having 1 to 30 carbon atoms, and M⁺ is selected from the group of the alkali metal ions and the group of the ammonium ions which are substituted by one or more alkyl and/or one or more hydroxyalkyl radicals, or corresponds to half an equivalent of an alkaline-earth metal ion.

For example, sodium isostearyl lactylate, for example the product Pathionic® ISL from the American Ingredients Company, is advantageous.

The betaines are advantageously selected from the group of the substances which are distinguished by the structural formula

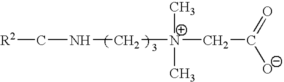

where R² denotes a branched or unbranched alkyl radical having 1 to 30 carbon atoms.

R² particularly advantageously denotes a branched or unbranched alkyl radical having 6 to 12 carbon atoms.

For example, capramidopropylbetaine, for example the product Tego® Betain 810 from Th. Goldschmidt AG, is advantageous.

A coconut amphoacetate which is advantageous for the purposes of the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological compositions according to the invention are applied in sufficient amount to the skin in the usual manner for cosmetics.

Cosmetic and dermatological compositions according to the invention may exist in various forms. Thus, they may be, for example, a solution, a waterfree composition, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoins in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

Co-emulsifiers which are advantageous for the purposes of the invention are, for example, O/W emulsifiers, principally from the group of the substances having HLB values of 11-16, very particularly advantageously having HLB values of 14.5-15.5, so long as the 0/W emulsifiers have saturated radicals R and R′. If the 0/W emulsifiers have unsaturated radicals R and/or R′ or in the case of isoalkyl derivatives, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

Optional W/O emulsifiers, but ones which may nevertheless be advantageous for the purposes of the invention can be the following:

fatty alcohols having 8 to 30 carbon atoms, monoglycerol esters of saturated and/or unsaturated, branched and/or unbranched alkanecarboxylic acids having a chain length of 8 to 24, in particular 12-18, C atoms, diglycerol esters of saturated and/or unsaturated, branched and/or unbranched alkanecarboxylic acids having a chain length of 8 to 24, in particular 12-18, C atoms, monoglycerol ethers of saturated and/or unsaturated, branched and/or unbranched alcohols having a chain length of 8 to 24, in particular 12-18, C atoms, diglycerol ethers of saturated and/or unsaturated, branched and/or unbranched alcohols having a chain length of 8 to 24, in particular 12-18, C atoms, propylene glycol esters of saturated and/or unsaturated, branched and/or unbranched alkanecarboxylic acids having a chain length of 8 to 24, in particular 12-18, C atoms, and sorbitan esters of saturated and/or unsaturated, branched and/or unbranched alkanecarboxylic acids having a chain length of 8 to 24, in particular 12-18, C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG 30 dipolyhydroxystearate.

The agents or compositions described are particularly suitable for protecting human skin against UV radiation, ageing processes and against oxidative stress, i.e. against damage by free radicals. In this connection, they are in the various administration forms usually used for this application. For example, the composition may be, in particular, in the form of a lotion or emulsion, such as in the form of a cream or milk (O/W, W/O, O/W/O, W/O/W), in the form of oily-alcoholic, oily-aqueous or aqueous-alcoholic gels or solutions, in the form of solid sticks or may be formulated as an aerosol.

The composition may comprise cosmetic adjuvants which are usually used in this type of composition, such as, for example, thickeners, softeners, moisturisers, surfactants, emulsifiers, preservatives, antifoams, perfumes, waxes, lanolin, propellants, dyes and/or pigments which colour the composition itself or the skin, and other ingredients usually used in cosmetics.

The dyes used are preferably approved dyes which are listed in the Cosmetics Regulation, Annex 3, as positive list.

The preservatives used are preferably approved preservatives which are listed in the Cosmetics Regulation, Annex 6, as positive list or also anti-microbial pigments, as described, for example, in WO 2004/0092283 or WO 2004/091567.

Suitable preservatives are therefore also alkyl esters of p-hydroxybenzoic acid, hydantoin derivatives, propionate salts or a multiplicity of ammonium compounds.

Very particularly preferred preservatives are methylparaben, propylparaben, imidazolidinylurea, sodium dehydroxyacetate or benzyl alcohol. Preservatives are employed in amounts between 0.5 and 2% by weight.

Emollients or softeners are often incorporated into cosmetic compositions. They are preferably employed in 0.5 to 50% by weight, preferably between 5 and 30% by weight, based on the composition as a whole. In general, softeners can be classified in classes, such as, for example, the category of the esters, fatty acids or fatty alcohols, polyols, hydrocarbons and oils containing at least one amide structural unit.

Representative oils containing at least one amide structural unit together with their synthesis are described, in particular, in EP 1044676 and EP 0928608. A compound which is particularly preferably indicated is isopropyl N-lauroylsarcosinate, which is commercially available from Ajinomoto under the product name Eldew SL-205.

Of the esters, mono- or diesters can be selected. Examples in this respect are dibutyl adipate, diethyl sebacate, diisopropyl dimerate or dioctyl succinate. Branched fatty acid esters are, for example, 2-ethylhexyl myristate, isopropyl stearate or isostearyl palmitate. Tribasic esters are, for example, triisopropyl trilinoleate or trilauryl citrate. Straight-chain fatty acid esters are, for example, lauryl palmitate, myristyl lactate, oleyl erucate or stearyl oleate. Preferred esters are Coco-Caprylate/Caprate (=INCI name, these are esters of coconut fatty alcohols with saturated medium-chain fatty acids), propylene glycol myristyl ether acetate, diisopropyl adipate or cetyl octanoate.

Suitable fatty alcohols and acids are compounds which have 10 to 20 C atoms. Particularly preferred compounds are cetyl, myristyl, palmitic or stearic alcohol or acid.

Suitable polyols are linear or branched-chain alkylpolyhydroxyl compounds, for example propylene glycol, sorbitol or glycerol. However, it is also possible to employ polymeric polyols, for example polypropylene glycol or polyethylene glycol. Butylene glycol and propylene glycol are also particularly suitable compounds for enhancing the penetration capacity.

Examples of hydrocarbons as softeners are compounds which generally have 12 to 30 C atoms. Specific examples are arylalkyl benzoates, alkyl benzoates, mineral oils, Vaseline, squalenes or isoparaffins.

Further emollients or hydrophobicising agents are preferably C₁₂ to C₁₅ alkyl benzoates, dioctyl adipate, octyl stearate, octyldodecanol, hexyl Iaurate, octyldodecyl neopentanoate, cyclomethicone, dicaprylic ether, dimethicone, phenyltrimethicone, isopropyl myristate, caprylic/capric glycerides, propylene glycol dicaprylate/dicaprate or decyl oleate.

A further category of functional ingredients of cosmetic compositions are thickeners. Thickeners are generally employed in amounts between 0.1 and 20% by weight, preferably between 0.5 and 10% by weight, based on the total amount. Examples of these compounds are crosslinked polyacrylate materials, commercially available from B.F. Goodrich Company under the trade name Carbopol. It is also possible to use thickeners such as xanthan gum, carrageenan gum, gelatine gum, karaya gum, pectin gum or carob seed flour.

Under certain circumstances, it is possible for a compound to be both a thickener and also a softener. Examples thereof are silicone gums (kinematic viscosity >10 centistokes), esters, such as, for example, glycerol stearate, or cellulose derivatives, for example hydroxypropylcellulose.

The dispersant or solubiliser used can be an oil, wax or other fatty substance, a lower monoalcohol or lower polyol or mixtures thereof. Particularly preferred monoalcohols or polyols include ethanol, i-propanol, propylene glycol, glycerol and sorbitol.

A preferred embodiment of the invention is an emulsion in the form of a protective cream or milk which comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The composition according to the invention may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a composition is formulated as an aerosol, the customary propellants, such as alkanes, fluoroalkanes and chlorofluoroalkanes, are generally used.

The present invention furthermore relates to a process for the preparation of a composition which is characterised in that one or more compounds of the formula I or Ia containing radicals as described above or at least one mixture, as described above, are mixed with a vehicle which is suitable for topical applications, for example a cosmetically, pharmaceutically or dermatologically suitable vehicle.

The same applies to the preparation of a food supplement, characterised in that one or more compounds of the formula I or Ia containing radicals as described above or at least one mixture, as described above, is mixed with a vehicle which is suitable for foods.

The compositions according to the invention can be prepared with the aid of techniques which are well known to the person skilled in the art.

The mixing can result in dissolution, emulsification or dispersion of the at least one compound of the formula I or Ia or the mixture, as described above, in the vehicle.

The properties of compounds of the formula I or Ia or the mixture, as described above, should likewise be regarded as positive for use in foods or as food supplements or as functional food. The further explanations given for foods also apply correspondingly to food supplements and functional food.

The foods encompass all materials which are suitable for consumption by animals or for consumption by humans, for example vitamins and provitamins thereof, fats, minerals or amino acids. The foods may be solid, but also liquid, i.e. in the form of a beverage.

The present invention accordingly furthermore relates to the use of at least one compound of the formula I or Ia or the mixture comprising at least one compound of the formula Ia-1 and at least one compound of the formula Ia-2 as food additive for human or animal nutrition, and to compositions which are foods or food supplements and comprise corresponding vehicles.

Foods for the purposes of the invention are, for example, also foods which originate from a single natural source, such as, for example, sugar, unsweetened juice, squash or purée of a single plant species, such as, for example, unsweetened apple juice (for example also a mixture of different types of apple juice), grapefruit juice, orange juice, apple compote, apricot squash, tomato juice, tomato sauce, tomato purée, etc. Further examples of foods which can be enriched in accordance with the present invention with one or more compounds of the formula I are corn or cereals from a single plant species and materials produced from plant species of this type, such as, for example, cereal syrup, rye flour, wheat flour or oat bran. Mixtures of foods of this type are also suitable, for example multivitamin preparations, mineral mixtures or sweetened juice. Further examples of foods which may be mentioned are food preparations, for example prepared cereals, biscuits, mixed drinks, foods prepared especially for children, such as yoghurt, diet foods, low-calorie foods or animal feeds.

The foods thus encompass all edible combinations of carbohydrates, lipids, proteins, inorganic elements, trace elements, vitamins, water or active metabolites of plants and animals.

The foods are preferably used orally, for example in the form of meals, pills, tablets, capsules, powders, syrup, solutions or suspensions.

The foods according to the invention can be prepared with the aid of techniques which are well known to the person skilled in the art.

Due to their action, compounds of the formula I are also suitable as medicament ingredient.

The invention is explained in greater detail below with reference to examples. The invention can be carried out throughout the scope claimed and is not restricted to the examples giver here.

### EXAMPLES

The compounds of the formula I or is are prepared in accordance with Scheme 1:

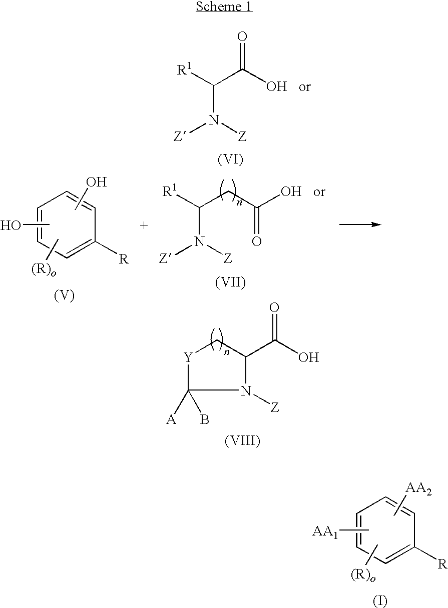

### Example 1

### Synthesis of 4-hexyl-3-hydroxyphenyl 2-pyrrolidone-5-carboxylate (1) and 2-hexyl-5-hydroxyphenyl 2-pyrrolidone-5-carboxylate (2)

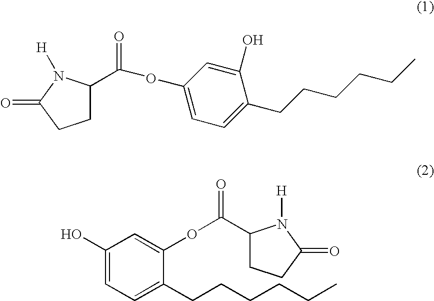

61.8 mmol of N,N′-dicyclohexylcarbodiimide and 4-dimethylaminopyridine (200 mg) are added to a solution of 4-hexylresorcinol (51.5 mmol) in 200 ml of methylene chloride. 61.8 mmol of (S)-(+2-pyrrolidone-5-carboxylic acid are then added, and the solution is stirred at room temperature for 18 h. The precipitate is filtered off, and the filtrate is freed from solvent. Subsequent chromatography gives a mixture of the two compounds in a ratio of ˜9:1[(1):(2)]. If the reaction is carried out at room temperature, the compounds are formed in a ratio of ˜1:1[(1):(2)].

The 9:1 mixture can also be separated into the individual components by column chromatography and/or recrystallisation.

4-Hexyl-3-hydroxyphenyl 2-pyrrolidinone-5-carboxylate (1)

1H NMR (DMSO-d6) δ 9.58 (s, 1H), 8.13 (s, 1H), 7.10 (d, J=8.2 Hz, 1H), 6.59 (d, J=8.2, 2.4 Hz, 1H), 4.46 (dd, J=9.2, 3.4 Hz, 1H), 2.54 (m, 3H), 2.25 (m, 3H), 1.55 (m, 2H), 1.32 (m, 6H), 0.90 (t, J=6.60 Hz, 3H).

4-Hexyl-3-hydroxyphenyl 2-pyrrolidinone-5-carboxylate (1) and 2-hexyl-5-hydroxyphenyl 2-pyrrolidinone-5-carboxylate (2) (9:1 mixture)

1H NMR (DMSO-d6) δ 9.58 (s, 1H-(1)+1H-(2)), 8.20 (s, 1H-(2)), 8.13 (s, 1H-(1)), 7.10 (d, J=8.2 Hz, 1H-(1)+1H-(2)), 6.68 (dd, J=8.3, 2.4 Hz, 1H-(2)), 6.63-6.50 (m, 2H-(1)+1H-(2)), 4.56-4.40 (m, 1H-(1)+1H-(2)), 2.54 (m, 3H-(1)+3H-(2)), 2.25 (m, 3H), 2.43-2.15 (m, 3H-(1)+3H-(2)), 1.65-1.42 (m, 2H-(1)+2H-(2)), 1.32 (m, 6H-(1)+6H-(2)), 0.90 (m, 3H-(1)+3H-(2)).

### Example 2

### Synthesis of 4-hexyl-3-hydroxyphenyl 4-dimethylaminobutyrate (3) and 2-hexyl-5-hydroxyphenyl 4-dimethylaminobutyrate (4)

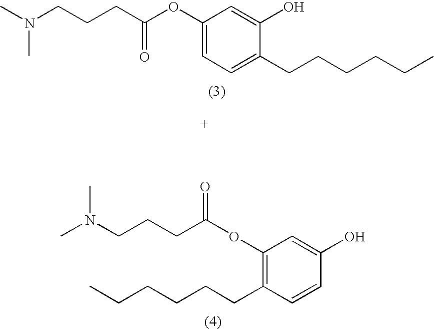

4-Hexylresorcinol and 4-dimethylaminobutyric acid are reacted analogously to Example 1 and reaction scheme 1.

### Example 3

### Synthesis of 4-hexyl-3-hydroxyphenyl dimethylaminoacetate (5) and 2-hexyl-5-hydroxyphenyl dimethylaminoacetate (6)

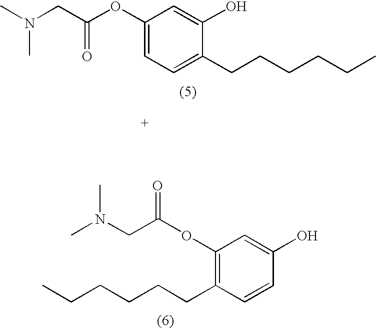

4-Hexylresorcinol and 4-dimethylaminoacetic acid are reacted analogously to Example 1 and reaction scheme 1.

### Example A

### Tyrosinase Assay

The action of the compounds I or Ia as skin lighteners is tested through their ability to inhibit the enzyme tyrosinase and thus to suppress melanin synthesis.

The inhibiting action of the compounds of the formula I or Ia against tyrosinase was assessed using tyrosinase from fungi and L-DOPA as substrate.

The compounds (1) and (2), as obtained in Example 1, and L-dopa are pre-incubated for 10 minutes at 25° C. in phosphate buffer (pH 6.8), and tyrosinase from fungi (16U) (Fluka) is subsequently added. The optical density of the samples is measured at 470 nm against a negative control (without active compound). Kojic acid and 4-hexylresorcinol are also tested as tyrosinase reference, i.e. positive control.

The results are shown in the following table, expressed in the IC₅₀ value (the concentration of the test compound at which 50% of the tyrosinase activity is inhibited).

TABLE
Test results of the tyrosinase assay

| Substance | IC₅₀ (μM) |
|---|---|
| 4-Hexyl-3-hydroxyphenyl | 19 |
| 2-pyrrolidinone-5-carboxylate (1) | |
| (1) + (2) [9:1 mixture] | 19 |
| Kojic acid | 12 |
| 4-Hexylresorcinol | 0.5 |

### Example B

### Depigmentation Activity on the Skin

The depigmentation activity of compounds of type I or Ia is tested in vitro using a reconstituted human tanned epidermis model (SkinEthic Laboratories).

The tanned epidermal tissues (11 days old, size 0.5 cm²) are treated daily for 4 days with 5 μl of a phosphate Puffer solution of the 9:1 mixture of the compounds 4-hexyl-3-hydroxyphenyl 2-pyrrolidinone-5-carboxylate (1) and 2-hexyl-5-hydroxyphenyl 2-pyrrolidinone-5-carboxylate (2).

In parallel, a negative control (epidermal tissues treated only with pure phosphate buffer) and a positive control are set up for comparison. All skin cultures are incubated at 37° C. for 6 days.

After the treatments, all epidermal tissues are investigated with respect to cell morphology and cell development ability by visual assessment. In addition, the melanin content is determined quantitatively.

### Example C

### Compositions

Illustrative formulations for cosmetric compositions which comprise compounds in accordance with Examples 1 to 3 are indicated below. In addition, the INCI names of the commercially available compounds are given.

UV-Pearl, OMC stands for the composition with the NCl name:

Water (for EU: Aqua), Ethylhexyl Methoxycinnamate, Silica, PVP, chlorophenesin, BHT; this composition is commercially available from Merck KGaA, Darmstadt, under the name Eusolex® UV Pearl™ OMC.

The other UV-Pearl products indicated in the tables each have an analogous composition, with OMC replaced by the UV filters indicated.

TABLE 1
W/O emulsions (data in % by weight)

| | 1-1 | 1-2 | 1-3 | 1-4 | 1-5 | 1-6 | 1-7 | 1-8 | 1-9 |
|---|---|---|---|---|---|---|---|---|---|
| Titanium Dioxide | | 2 | 5 | | | | | | |
| (1) + (2) [mixture] | 0.1 | 0.2 | 0.3 | 0.4 | 0.7 | | | | 0.5 |
| (3) + (4) [mixture] | | | | | | 0.1 | 0.2 | 0.3 | |
| Zinc oxide | | | | | | | | 5 | 2 |
| UV-Pearl, OMC | 30 | 15 | 15 | 15 | 15 | 15 | 15 | 15 | 15 |
| Polyglyceryl-3-Dimerate | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 |
| Caprylic/Capric | 7 | 7 | 7 | 7 | 7 | 7 | 7 | 7 | 7 |
| Triglyceride | | | | | | | | | |
| Hexyl Laurate | 4 | 4 | 4 | 4 | 4 | 4 | 4 | 4 | 4 |
| PVP/Eicosene Copolymer | 2 | 2 | 2 | 2 | 2 | 2 | 2 | 2 | 2 |
| Propylene Glycol | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 |
| Tocopherol | 0.5 | 0.5 | 0.5 | 0.5 | 0.5 | 0.5 | 0.5 | 0.5 | 0.5 |
| Tocopheryl Acetate | 0.5 | 0.5 | 0.5 | 0.5 | 0.5 | 0.5 | 0.5 | 0.5 | 0.5 |
| Cyclomethicone | 0.5 | 0.5 | 0.5 | 0.5 | 0.5 | 0.5 | 0.5 | 0.5 | 0.5 |
| Propylparaben | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 |
| Water | to 100 | to 100 | to 100 | to 100 | to 100 | to 100 | to 100 | to 100 | to 100 |

| | 1-11 | 1-12 | 1-13 | 1-14 | 1-15 | 1-16 | 1-17 | 1-18 |
|---|---|---|---|---|---|---|---|---|
| Titanium Dioxide | 3 | | 2 | | 3 | | 2 | 5 |
| Benzylidene malonate | | 1 | 0.5 | | | | | |
| polysiloxane | | | | | | | | |
| Methylene Bis-Benztriazolyl | 1 | 1 | 0.5 | | | | | |
| Tetramethylbutylphenol | | | | | | | | |
| (5) + (6) [mixture] | 0.1 | 0.2 | 0.3 | 0.5 | 0.7 | 0.2 | 0.4 | 0.2 |
| Polyglyceryl-3-Dimerate | 3 | 3 | 3 | 3 | | | | |
| Cera Alba | 0.3 | 0.3 | 0.3 | 0.3 | 2 | 2 | 2 | 2 |
| Hydrogenated Castor Oil | 0.2 | 0.2 | 0.2 | 0.2 | | | | |
| Paraffinium Liquidum | 7 | 7 | 7 | 7 | | | | |
| Caprylic/Capric Triglyceride | 7 | 7 | 7 | 7 | | | | |
| Hexyl Laurate | 4 | 4 | 4 | 4 | | | | |
| PVP/Eicosene Copolymer | 2 | 2 | 2 | 2 | | | | |
| Propylene Glycol | 4 | 4 | 4 | 4 | | | | |
| Magnesium Sulfate | 0.6 | 0.6 | 0.6 | 0.6 | | | | |
| Tocopherol | 0.5 | 0.5 | 0.5 | 0.5 | | | | |
| Tocopheryl Acetate | 0.5 | 0.5 | 0.5 | 0.5 | 1 | 1 | 1 | 1 |
| Cyclomethicone | 0.5 | 0.5 | 0.5 | 0.5 | | | | |
| Propylparaben | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 |
| Dicocoyl Pentyerythrityl Citrate | | | | | 6 | 6 | 6 | 6 |
| (and) Sorbitan Sesquioleate | | | | | | | | |
| (and) Cera Alba (and) Aluminium | | | | | | | | |
| Stearate | | | | | | | | |
| PEG-7 Hydrogenated Castor Oil | | | | | 1 | 1 | 1 | 1 |
| Zinc Stearate | | | | | 2 | 2 | 2 | 2 |
| Oleyl Erucate | | | | | 6 | 6 | 6 | 6 |
| Decyl Oleate | | | | | 6 | 6 | 6 | 6 |
| Dimethicone | | | | | 5 | 5 | 5 | 5 |
| Tromethamine | | | | | 1 | 1 | 1 | 1 |
| Glycerin | | | | | 5 | 5 | 5 | 5 |
| Allantoin | | | | | 0.2 | 0.2 | 0.2 | 0.2 |
| Water | to 100 | to 100 | to 100 | to 100 | to 100 | to 100 | to 100 | to 100 |

TABLE 2
O/W emulsions, data in % by weight

| | 2-1 | 2-2 | 2-3 | 2-4 | 2-5 | 2-6 | 2-7 | 2-8 | 2-9 | 2-10 |
|---|---|---|---|---|---|---|---|---|---|---|
| Titanium Dioxide | | 2 | 5 | | | | | | | 3 |
| Methylene Bis- | | | | | | 1 | 2 | 1 | | |
| Benztriazolyl | | | | | | | | | | |
| Tetramethylbutylphenol | | | | | | | | | | |
| (1) + (2) [mixture] | 0.1 | 0.2 | | 0.2 | 0.2 | 0.25 | | 0.3 | 0.2 | 0.1 |
| (3) + (4) [mixture] | 0.2 | 0.1 | 0.3 | 0.2 | 0.1 | 0.25 | 0.2 | 0.2 | 0.1 | 0.2 |
| (5) + (6) [mixture] | 0.1 | 0.1 | 0.1 | | 0.3 | | 0.1 | | 0.2 | 0.3 |
| 4-Methylbenzylidene | 2 | | 3 | | 4 | | 3 | | 2 | |
| Camphor | | | | | | | | | | |
| BMDBM | 1 | 3 | | 3 | 3 | | 3 | 3 | 3 | |
| Stearyl Alcohol (and) | 3 | 3 | 3 | 3 | 3 | 3 | 3 | 3 | 3 | 3 |
| Steareth-7 (and) | | | | | | | | | | |
| Steareth-10 | | | | | | | | | | |
| Glyceryl Stearate (and) | 3 | 3 | 3 | 3 | 3 | 3 | 3 | 3 | 3 | 3 |
| Ceteth-20 | | | | | | | | | | |
| Glyceryl Stearate | 3 | 3 | 3 | 3 | 3 | 3 | 3 | 3 | 3 | 3 |
| Microwax | 1 | 1 | 1 | 1 | 1 | 1 | 1 | 1 | 1 | 1 |
| Cetearyl Octanoate | 11.5 | 11.5 | 11.5 | 11.5 | 11.5 | 11.5 | 11.5 | 11.5 | 11.5 | 11.5 |
| Caprylic/Capric | 6 | 6 | 6 | 6 | 6 | 6 | 6 | 6 | 6 | 6 |
| Triglyceride | | | | | | | | | | |
| Oleyl Oleate | 6 | 6 | 6 | 6 | 6 | 6 | 6 | 6 | 6 | 6 |
| Propylene Glycol | 4 | 4 | 4 | 4 | 4 | 4 | 4 | 4 | 4 | 4 |
| Glyceryl Stearate SE | | | | | | | | | | |
| Stearic Acid | | | | | | | | | | |
| Persea Gratissima | | | | | | | | | | |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Tromethamine | | | 1.8 | | | | | | | |
| Glycerin | | | | | | | | | | |
| Water | to 100 | to 100 | to 100 | to 100 | to 100 | to 100 | to 100 | to 100 | to 100 | to 100 |

| | 2-11 | 2-12 | 2-13 | 2-14 | 2-15 | 2-16 | 2-17 | 2-18 |
|---|---|---|---|---|---|---|---|---|
| Titanium Dioxide | 3 | | 2 | | | | 2 | 5 |
| Benzylidene malonate | | 1 | 0.5 | | | | | |
| polysiloxane | | | | | | | | |
| Methylene Bis- | 1 | 1 | 0.5 | | | | | |
| Benztriazolyl | | | | | | | | |
| Tetramethylbutylphenol | | | | | | | | |
| (1) + (2) [mixture] | 5 | 5 | 5 | 5 | 5 | 5 | 5 | 5 |
| Zinc oxide | | | 2 | | | | | |
| UV-Pearl, OMC | 15 | 15 | 15 | 30 | 30 | 30 | 15 | 15 |
| 4-Methylbenzylidene | | | | 3 | | | | |
| Camphor | | | | | | | | |
| BMDBM | | | | 1 | | | | |
| Phenylbenzimidazole | | | | | 4 | | | |
| Sulfonic Acid | | | | | | | | |
| Stearyl Alcohol (and) | 3 | 3 | 3 | 3 | | | | |
| Steareth-7 (and) | | | | | | | | |
| Steareth-10 | | | | | | | | |
| Glyceryl Stearate (and) | 3 | 3 | 3 | 3 | | | | |
| Ceteth-20 | | | | | | | | |
| Glyceryl Stearate | 3 | 3 | 3 | 3 | | | | |
| Microwax | 1 | 1 | 1 | 1 | | | | |
| Cetearyl Octanoate | 11.5 | 11.5 | 11.5 | 11.5 | | | | |
| Caprylic/Capric | 6 | 6 | 6 | 6 | 14 | 14 | 14 | 14 |
| Triglyceride | | | | | | | | |
| Oleyl Oleate | 6 | 6 | 6 | 6 | | | | |
| Propylene Glycol | 4 | 4 | 4 | 4 | | | | |
| Glyceryl Stearate SE | | | | | 6 | 6 | 6 | 6 |
| Stearic Acid | | | | | 2 | 2 | 2 | 2 |
| Persea Gratissima | | | | | 8 | 8 | 8 | 8 |
| Propylparaben | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 |
| Tromethamine | | | | | 1.8 | | | |
| Glycerin | | | | | 3 | 3 | 3 | 3 |
| Water | to 100 | to 100 | to 100 | to 100 | to 100 | to 100 | to 100 | to 100 |

TABLE 3
Gels, data in % by weight

| | 3-1 | 3-2 | 3-3 | 3-4 | 3-5 | 3-6 | 3-7 | 3-8 | 3-9 | 3-10 |
|---|---|---|---|---|---|---|---|---|---|---|
| Titanium Dioxide | | 2 | 5 | | | | | | | 3 |
| (1) + (2) [mixture] | | | | 0.1 | 0.2 | | | | 0.1 | 0.1 |
| (3) + (4) [mixture] | 0.1 | 0.2 | | 0.1 | | 0.1 | | 0.5 | 0.2 | |
| (5) + (6) [mixture] | 0.2 | 0.1 | 0.3 | 0.5 | 0.1 | 0.2 | 0.3 | 0.2 | 0.2 | 0.5 |
| Compound (1) | 0.1 | 0.25 | 0.4 | | 0.1 | | 0.4 | | 0.1 | 0.1 |
| Benzylidene malonate | | | 1 | 1 | 2 | | | | 1 | 1 |
| polysiloxane | | | | | | | | | | |
| Methylene Bis- | | 1 | | | | 1 | 2 | 1 | | |
| Benztriazolyl | | | | | | | | | | |
| Tetramethylbutylphenol | | | | | | | | | | |
| Zinc oxide | | | | 2 | | | | 5 | 2 | |
| UV-Pearl, Ethylhexyl | 30 | 15 | 15 | 15 | 15 | 15 | 15 | 15 | 15 | 15 |
| Methoxycinnamat | | | | | | | | | | |
| 4-Methylbenzylidene | | | | | 2 | | | | | |
| Camphor | | | | | | | | | | |
| Butylmethoxydibenzoyl | | 1 | | | | | | | | |
| methane | | | | | | | | | | |
| Phenylbenzimidazole | | | 4 | | | | | | | |
| Sulfonic Acid | | | | | | | | | | |
| Prunus Dulcis | 5 | 5 | 5 | 5 | 5 | 5 | 5 | 5 | 5 | 5 |
| Tocopheryl Acetate | 0.5 | 0.5 | 0.5 | 0.5 | 0.5 | 0.5 | 0.5 | 0.5 | 0.5 | 0.5 |
| Caprylic/Capric | 3 | 3 | 3 | 3 | 3 | 3 | 3 | 3 | 3 | 3 |
| Triglyceride | | | | | | | | | | |
| Octyldodecanol | 2 | 2 | 2 | 2 | 2 | 2 | 2 | 2 | 2 | 2 |
| Decyl Oleate | 2 | 2 | 2 | 2 | 2 | 2 | 2 | 2 | 2 | 2 |
| PEG-8 (and) Tocopherol | 0.05 | 0.05 | 0.05 | 0.05 | 0.05 | 0.05 | 0.05 | 0.05 | 0.05 | 0.05 |
| (and) Ascorbyl Palmitate | | | | | | | | | | |
| (and) Ascorbic Acid | | | | | | | | | | |
| (and) Citric Acid | | | | | | | | | | |
| Sorbitol | 4 | 4 | 4 | 4 | 4 | 4 | 4 | 4 | 4 | 4 |
| Polyacrylamide (and) | 3 | 3 | 3 | 3 | 3 | 3 | 3 | 3 | 3 | 3 |
| C13-14 Isoparaffin (and) | | | | | | | | | | |
| Laureth-7 | | | | | | | | | | |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Tromethamine | | | 1.8 | | | | | | | |
| Water | to 100 | to 100 | to 100 | to 100 | to 100 | to 100 | to 100 | to 100 | to 100 | to 100 |

1. A compound of formula I 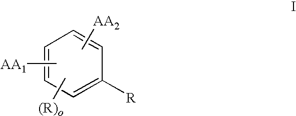
where
AA₁ and AA₂ each, independently of one another, denote OH or a radical of a proteinogenic or non-proteinogenic amino acid,
R in each case, independently of one another, optionally substituted, denotes a linear or branched alkyl group having 1 to 12 C atoms, cycloalkyl having 3 to 9 C atoms or cycloalkenyl having 5 to 9 C atoms,
o is 0, 1 or 2,
with the proviso that AA₁ and AA₂ are not simultaneously OH,
or a salt or solvate thereof.

2. A compound according to claim 1, wherein the radical R is of formula II, III or IV: 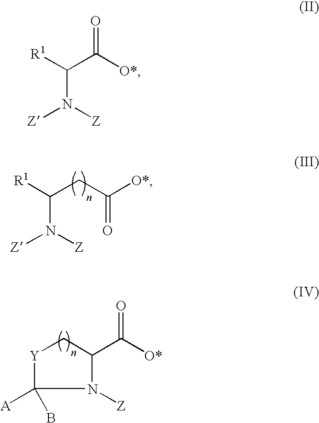
where
Z and Z′ each independently denote H, R, OCOCH₃, OCOC₆H₅, CH₂C₆H₅ or OCOC(CH₃)₃,
R¹ denotes H, or a linear or branched alkyl group having 1 to 30 C atoms, optionally substituted by OH, OR, SH, SR, NH₂, NHR, NR₂, substituted or unsubstituted guanidinium, (NR₃)⁺, COOH, COOR, CONH₂, CONHR, CONR₂, COR, Ar or Het,
R in each case, independently of one another, optionally substituted, denotes a linear or branched alkyl group having 1 to 12 C atoms, cycloalkyl having 3 to 9 C atoms or cycloalkenyl having 5 to 9 C atoms,
Y denotes —CH₂— or —NZ—,
A and B each, independently of one another, denote H, R or OH, or A and B, together with the C atom to which they are linked, may form a carbonyl group,
n is 1, 2 or 3,
Ar denotes phenyl which is unsubstituted or mono-, di- or trisubstituted by R, OH or OR,
Het denotes a saturated or unsaturated mono- or bicyclic heterocyclic radical having 5 to 13 ring members, where 1, 2 or 3 N and/or 1 or 2 S or O atoms may be present, and the heterocyclic radical may be mono- or polysubstituted by R, OH or OR, and
double bonds may also be present in the ring in formula IV.

3. A compound according to claim 1, which is of formula Ia 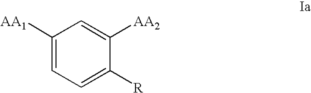
where AA₁, AA₂ and R have a meaning indicated for the compound of formula I.

4. A compound according to claim 2, wherein AA₁ is of formula II and AA₂ denotes OH, or AA₂ is of formula II and AA₁ denotes OH.

5. A compound according to claim 2, wherein AA₁ is of formula III and AA₂ denotes OH, or AA₂ is of formula III and AA₁ denotes OH.

6. A compound according to claim 2, wherein AA₁ is of formula IV and AA₂ denotes OH, or AA₂ is of formula IV and AA₁ denotes OH.

7. A compound according to claim 1, which is:
4-hexyl-3-hydroxyphenyl 2-pyrrolidinone-5-carboxylate,
2-hexyl-5-hydroxyphenyl 2-pyrrolidinone-5-carboxylate,
4-hexyl-3-hydroxyphenyl 4-dimethylaminobutyrate,
2-hexyl-5-hydroxyphenyl 4-dimethylaminobutyrate,
4-hexyl-3-hydroxyphenyl dimethylaminoacetate, or
2-hexyl-5-hydroxyphenyl dimethylaminoacetate,
or a salt or solvate thereof.

8. A mixture comprising
at least one compound of the formula Ia-1 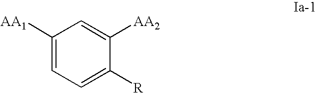
and at least one compound of the formula Ia-2 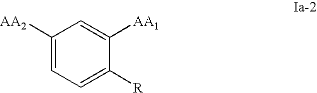
in which
AA₁, AA₂ and R are identical for both compounds of formulae Ia-1 and Ia-2 whereby AA₁ in the compound of formula Ia-1 is identical to AA₁ in the compound of formula Ia-2, AA₂ in the compound of formula Ia-1 is identical to AA₂ in the compound of formula Ia-2, and R in the compound of formula Ia-1 is identical to R in the compound of formula Ia-2, and
wherein
R is optionally substituted, and denotes a linear or branched alkyl group having 1 to 12 C atoms, cycloalkyl having 3 to 9 C atoms or cycloalkenyl having 5 to 9 C atoms, and
AA₁ is of formula II or III or IV and AA₂ denotes OH, or AA₂ is of formula II or III or IV and AA₁ denotes OH, 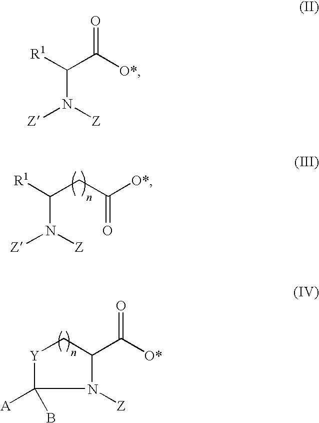
where
Z and Z′ each independently denote H, R, OCOCH₃, OCOC₆H₅, CH₂C₆H₅ or OCOC(CH₃)₃,
R¹ denotes H, or a linear or branched alkyl group having 1 to 30 C atoms, optionally, substituted by OH, OR, SH, SR, NH₂, NHR, NR₂, substituted or unsubstituted guanidinium, (NR₃)⁺, COOH, COOR, CONH₂, CONHR, CONR₂, COR, Ar or Het,
R in each case, independently of one another, optionally substituted, denotes a linear or branched alkyl group having 1 to 12 C atoms, cycloalkyl having 3 to 9 C atoms or cycloalkenyl having 5 to 9 C atoms,
Y denotes —CH₂— or —NZ—,
A and B each, independently of one another, denote H, R or OH, or A and B, together with the C atom to which they are linked, may form a carbonyl group,
n is 1, 2 or 3,
Ar denotes phenyl which is unsubstituted or mono-, di- or trisubstituted by R, OH or OR,
Het denotes a saturated or unsaturated mono- or bicyclic heterocyclic radical having 5 to 13 ring members, where 1, 2 or 3 N and/or 1 or 2 S or O atoms may be present, and the heterocyclic radical may be mono- or polysubstituted by R, OH or OR,
or a salt or solvate thereof.

9. A mixture according to claim 8, further comprising at least one compound of the formula Va 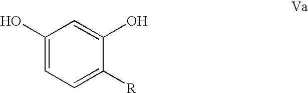
where R is identical in the compound of formula Va with the R in the compounds of formulae Ia-1 and Ia-2,
or a salt or solvate thereof.

10. A mixture comprising
at least one compound of the formula Va 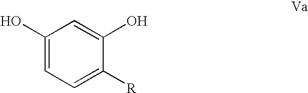
and only one of
at least one compound of the formula Ia-1 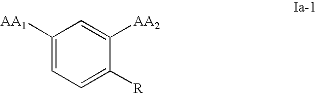
or
at least one compound of the formula Ia-2 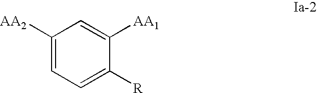
in which
R is identical in the compound of formula Va with the R in the compound of formula Ia-1 or Ia-2,
wherein
AA₁ and AA₂ each, independently of one another, denote OH or a radical of a proteinogenic or non-proteinogenic amino acid,
R in each case, independently of one another, optionally substituted, denotes a linear or branched alkyl group having 1 to 12 C atoms, cycloalkyl having 3 to 9 C atoms or cycloalkenyl having 5 to 9 C atoms,
with the proviso that AA₁ and AA₂ are not simultaneously OH,
or a salt or solvate thereof.

11. A process for preparing a compound of formula I according to claim 1 or a salt or solvate thereof, comprising
(a) reacting a compound of the formula V or Va 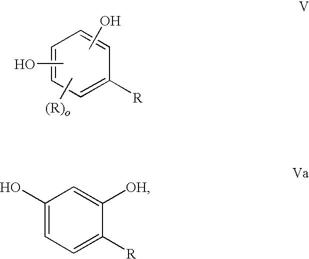
in which R and o have a meaning indicated for the compound of formula I,
with a
proteinogenic or non-proteinogenic amino acid, or with a compound of formula VI 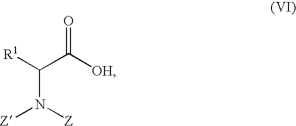
of formula VII 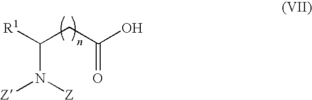
or
of formula VIII 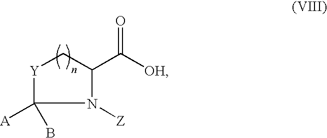
where
Z and Z′ each independently denote H, R, OCOCH₃, OCOC₆H₅, CH₂C₆H₅ or OCOC(CH₃)₃,
R¹ denotes H, or a linear or branched alkyl group having 1 to 30 C atoms, optionally OH, OR, SH, SR, NH₂, NHR, NR₂, substituted or unsubstituted guanidinium, (NR₃)⁺, COOH, COOR, CONH₂, CONHR, CONR₂, COR, Ar or Het,
R in each case, independently of one another, optionally substituted, denotes a linear or branched alkyl group having 1 to 12 C atoms, cycloalkyl having 3 to 9 C atoms or cycloalkenyl having 5 to 9 C atoms,
Y denotes —CH₂— or —NZ—,
A and B each, independently of one another, denote H, R or OH, or A and B, together with the C atom to which they are linked, may form a carbonyl group,
n is 1, 2 or 3,
Ar denotes phenyl which is unsubstituted or mono-, di- or trisubstituted by R, OH or OR,
Het denotes a saturated or unsaturated mono- or bicyclic heterocyclic radical having 5 to 13 ring members, where 1, 2 or 3 N and/or 1 or 2 S or O atoms may be present, and the heterocyclic radical may be mono- or polysubstituted by R, OH or OR,
and
(b) optionally
a basic or acidic compound of formula I is converted into one of its salts or solvates by treatment with an acid or base or solvent.

12. A composition comprising
at least one vehicle which is suitable for topical applications,
and
a compound of formula I 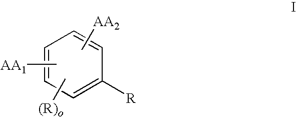
where
AA₁ and AA₂ each, independently of one another, denote OH or a radical of a proteinogenic or non-proteinogenic amino acid,
R in each case, independently of one another, optionally substituted, denotes a linear or branched alkyl group having 1 to 12 C atoms, cycloalkyl having 3 to 9 C atoms or cycloalkenyl having 5 to 9 C atoms,
o is 0, 1 or 2,
with the proviso that AA₁ and AA₂ are not simultaneously OH,
or a salt or solvate thereof,
or
a mixture comprising
at least one compound of the formula Ia-1 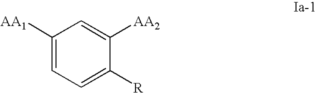
and
at least one compound of the formula Ia-2 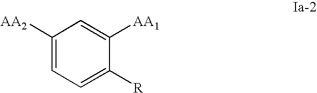
in which
AA₁, AA₂ and R are identical for both compounds of formulae Ia-1 and Ia-2 whereby AA₁ in the compound of formula Ia-1 is identical to AA₁ in the compound of formula Ia-2, AA₂ in the compound of formula Ia-1 is identical to AA₂ in the compound of formula Ia-2, and R in the compound of formula Ia-1 is identical to R in the compound of formula Ia-2, and
wherein
R is optionally substituted, and denotes a linear or branched alkyl group having 1 to 12 C atoms, cycloalkyl having 3 to 9 C atoms or cycloalkenyl having 5 to 9 C atoms, and
AA₁ is of formula II or III or IV and AA₂ denotes OH or AA₂ is of formula II or III or IV and AA₁ denotes OH, 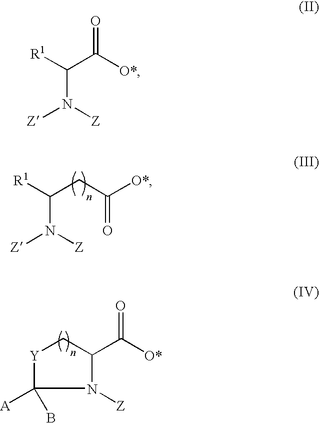
where
Z and Z′ each independently denote H, R, OCOCH₃, OCOC₆H₅, CH₂C₆H₅ or OCOC(CH₃)₃,
R¹ denotes H, or a linear or branched alkyl group having 1 to 30 C atoms, optionally substituted by OH, OR, SH, SR, NH₂, NHR, NR₂, substituted or unsubstituted guanidinium, (NR₃)⁺, COOH, COOR, CONH₂, CONHR, CONR₂, COR, Ar or Het,
R in each case, independently of one another, optionally substituted, denotes a linear or branched alkyl group having 1 to 12 C atoms, cycloalkyl having 3 to 9 C atoms or cycloalkenyl having 5 to 9 C atoms,
Y denotes —CH₂— or —NZ—,
A and B each, independently of one another, denote H, R or OH, or A and B, together with the C atom to which they are linked, may form a carbonyl group,
n is 1, 2 or 3,
Ar denotes phenyl which is unsubstituted or mono-, di- or trisubstituted by R, OH or OR,
Het denotes a saturated or unsaturated mono- or bicyclic heterocyclic radical having 5 to 13 ring members, where 1, 2 or 3 N and/or 1 or 2 S or O atoms may be present, and the heterocyclic radical may be mono- or polysubstituted by R, OH or OR,
or a salt or solvate thereof,
or
a mixture comprising
at least one compound of the formula Va 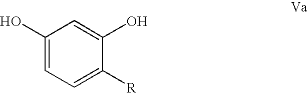
and only one of
at least one compound of the formula Ia-1 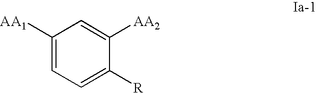
or
at least one compound of the formula Ia-2 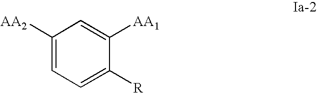
in which
R is identical in the compound of formula Va with the R in the compound of formula Ia-1 or Ia-2,
wherein
AA₁ and AA₂ each, independently of one another, denote OH or a radical of a proteinogenic or non-proteinogenic amino acid,
R in each case, independently of one another, optionally substituted, denotes a linear or branched alkyl group having 1 to 12 C atoms, cycloalkyl having 3 to 9 C atoms or cycloalkenyl having 5 to 9 C atoms,
with the proviso that AA₁ and AA₂ are not simultaneously OH,
or a salt or solvate thereof.

13. A composition according to claim 12, which comprises one or more compounds of formula I or a mixture comprising at least one compound of the formula Ia-1 and at least one compound of the formula Ia-2 or a mixture comprising at least one compound of the formula Va and at least one compound of the formula Ia-1 and/or at least one compound of the formula Ia-2 in an amount of 0.01 to 20% by weight, based on the total weight of the composition.

14. A composition according to claim 12, further comprising one or more antioxidants and/or one or more vitamins.

15. A composition according to claim 12, further comprising one or more UV filters.

16. A composition according to claim 12, further comprising at least one further compound having skin-lightening activity.

17. A composition according to claim 16, wherein the further compound having skin-lightening activity is selected from the group consisting of hydroquinone, kojic acid, arbutin, aloesin and rucinol.

18. A process for preparing composition according to claim 12, comprising mixing together one or more compounds of formula I or a mixture comprising at least one compound of the formula Ia-1 and at least one compound of the formula Ia-2 or a mixture comprising at least one compound of the formula Va and at least one compound of the formula Ia-1 and/or at least one compound of the formula Ia-2 with a vehicle which is suitable for topical applications.

19. A method for lightening human skin, or for inhibiting tyrosinase or for the prophylaxis or treatment of a pigment defect, hyperpigmentation, freckles, age spot, sun spot or environmentally induced skin aging, comprising administering to a subject
a compound of formula I 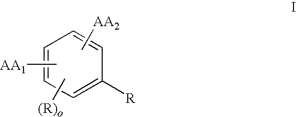
where
AA₁ and AA₂ each, independently of one another, denote OH or a radical of a proteinogenic or non-proteinogenic amino acid,
R in each case, independently of one another, optionally substituted, denotes a linear or branched alkyl group having 1 to 12 C atoms, cycloalkyl having 3 to 9 C atoms or cycloalkenyl having 5 to 9 C atoms,
o is 0, 1 or 2,
with the proviso that AA₁ and AA₂ are not simultaneously OH,
or a salt or solvate thereof,
or
a mixture comprising at least one compound of the formula Ia-1 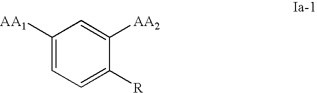
and at least one compound of the formula Ia-2 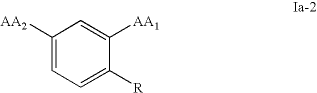
in which
AA₁, AA₂ and R are identical for both compounds of formulae Ia-1 and Ia-2 whereby AA₁ in the compound of formula Ia-1 is identical to AA₁, in the compound of formula Ia-2, AA₂ in the compound of formula Ia-1 is identical to AA₂ in the compound of formula Ia-2, and R in the compound of formula Ia-1 is identical to R in the compound of formula Ia-2, and
wherein
R is optionally substituted, and denotes a linear or branched alkyl group having 1 to 12 C atoms, cycloalkyl having 3 to 9 C atoms or cycloalkenyl having 5 to 9 C atoms, and
AA₁ is of formula II or III or IV and AA₂ denotes OH, or AA₂ is of formula II or III or IV and AA₁ denotes OH, 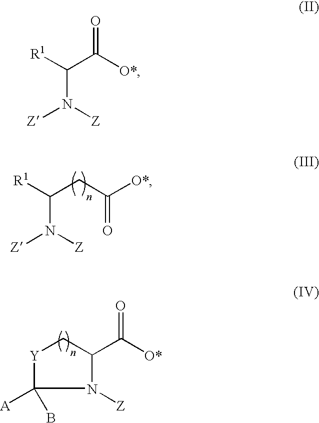
where
Z and Z′ each independently denote H, R, OCOCH₃, OCOC₆H₅, CH₂C₆H₅ or OCOC(CH₃)₃,
R¹ denotes H, or a linear or branched alkyl group having 1 to 30 C atoms, optionally substituted by OH, OR, SH, SR, NH₂, NHR, NR₂, substituted or unsubstituted guanidinium, (NR₃)⁺, COOH, COOR, CONH₂, CONHR, CONR₂, COR, Ar or Het,
R in each case, independently of one another, optionally substituted, denotes a linear or branched alkyl group having 1 to 12 C atoms, cycloalkyl having 3 to 9 C atoms or cycloalkenyl having 5 to 9 C atoms,
Y denotes —CH₂— or —NZ—,
A and B each, independently of one another, denote H, R or OH, or A and B, together with the C atom to which they are linked, may form a carbonyl group,
n is 1, 2 or 3,
Ar denotes phenyl which is unsubstituted or mono-, di- or trisubstituted by R, OH or OR,
Het denotes a saturated or unsaturated mono- or bicyclic heterocyclic radical having 5 to 13 ring members, where 1, 2 or 3 N and/or 1 or 2 S or O atoms may be present and the heterocyclic radical may be mono- or polysubstituted by R, OH or OR,
or a salt or solvate thereof,
or
a mixture comprising
at least one compound of the formula Va 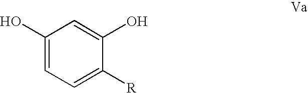
and only one of
at least one compound of the formula Ia-1 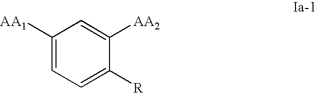
or
at least one compound of the formula Ia-2 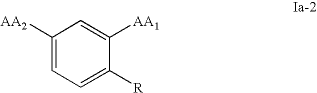
in which
R is identical in the compound of formula Va with the R in the compound of formula Ia-1 or Ia-2,
wherein
AA₁ and AA₂ each, independently of one another, denote OH or a radical of a proteinogenic non-proteinogenic
R in each case, independently of one another, optionally substituted, denotes a linear or branched alkyl group having 1 to 12 C atoms, cycloalkyl having 3 to 9 C atoms or cycloalkenyl having 5 to 9 C atoms,
with the proviso that AA₁ and AA₂ are not simultaneously OH,
or a salt or solvate thereof.

20. A method according to claim 19, which is for inhibiting tyrosinase.

21. A method according to claim 19, which is for the prophylaxis and/or treatment of a pigment defect, hyperpigmentation, freckles, age spot, sun spot or environmentally induced skin aging.

22. A method according to claim 19, which is for lightening human skin.

23. A compound according to claim 1, wherein R in each case, independently of one another, denotes a linear or branched alkyl group having 1 to 12 C atoms, cycloalkyl having 3 to 9 C atoms or cycloalkenyl having 5 to 9 C atoms.

24. A mixture according to claim 10, wherein
AA₁ is of formula II or III or IV and AA₂ denotes OH, or AA₂ is of formula II or III or IV and AA₁ denotes OH, 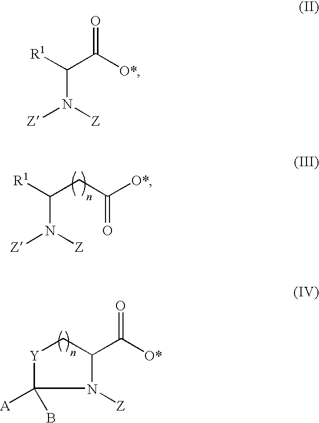
where
Z and Z′ each independently denote H, R, OCOCH₃, OCOC₆H₅, CH₂C₆H₅ or OCOC(CH₃)₃,
R¹ denotes H, or a linear or branched alkyl group having 1 to 30 C atoms, optionally substituted by OH, OR, SH, SR, NH₂, NHR, NR₂, substituted or unsubstituted guanidinium, (NR₃)⁺, COOH, COOR, CONH₂, CONHR, CONR₂, COR, Ar or Het,
R in each case, independently of one another, optionally substituted, denotes a linear or branched alkyl group having 1 to 12 C atoms, cycloalkyl having 3 to 9 C atoms or cycloalkenyl having 5 to 9 C atoms,
Y denotes —CH₂— or —NZ—,
A and B each, independently of one another, denote H, R or OH, or A and B, together with the C atom to which they are linked, may form a carbonyl group,
n is 1, 2 or 3,
Ar denotes phenyl which is unsubstituted or mono-, di- or trisubstituted by R, OH or OR,
Het denotes a saturated or unsaturated mono- or bicyclic heterocyclic radical having 5 to 13 ring members, where 1, 2 or 3 N and/or 1 or 2 S or O atoms may be present, and the heterocyclic radical may be mono- or polysubstituted by R, OH or OR.

25. A composition according to claim 12, wherein
in the mixture comprising
at least one compound of the formula Va
and only one of
at least one compound of the formula Ia-1 or
at least one compound of the formula Ia-2,
AA₁ is of formula II or III or IV and AA₂ denotes OH, or AA₂ is of formula II or III or IV and AA₁ denotes OH, 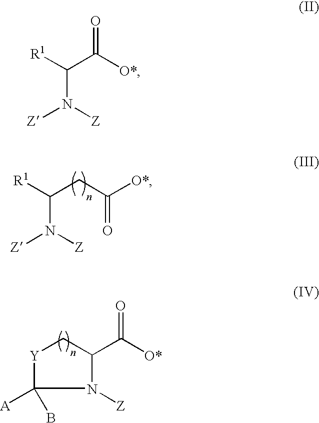
where
Z and Z′ each independently denote H, R, OCOCH₃, OCOC₆H₅, CH₂C₆H₅ or OCOC(CH₃)₃,
R¹ denotes H, or a linear or branched alkyl group having 1 to 30 C atoms, optionally substituted by OH, OR, SH, SR, NH₂, NHR, NR₂, substituted or unsubstituted guanidinium, (NR₃)⁺, COOH, COOR, CONH₂, CONHR, CONR₂, COR, Ar or Het,
R in each case, independently of one another, optionally substituted, denotes a linear or branched alkyl group having 1 to 12 C atoms, cycloalkyl having 3 to 9 C atoms or cycloalkenyl having 5 to 9 C atoms,
Y denotes —CH₂— or —NZ—,
A and B each, independently of one another, denote H, R or OH, or A and B, together with the C atom to which they are linked, may form a carbonyl group,
n is 1, 2 or 3,
Ar denotes phenyl which is unsubstituted or mono-, di- or trisubstituted by R, OH or OR,
Het denotes a saturated or unsaturated mono- or bicyclic heterocyclic radical having 5 to 13 ring members, where 1, 2 or 3 N and/or 1 or 2 S or O atoms may be present, and the heterocyclic radical may be mono- or polysubstituted by R, OH or OR.

26. A method according to claim 19, wherein
in the mixture comprising
at least one compound of the formula Va
and only one of
at least one compound of the formula Ia-1 or
at least one compound of the formula Ia-2,
AA₁ is of formula II or III or IV and AA₂ denotes OH, or AA₂ is of formula II or III or IV and AA₁ denotes OH, 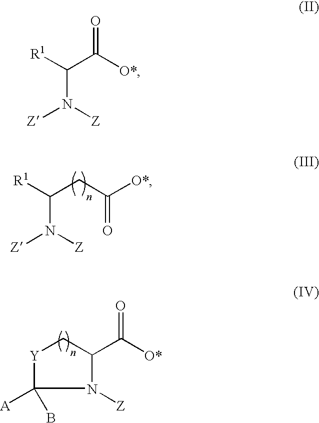
where
Z and Z′ each independently denote H, R, OCOCH₃, OCOC₆H₅, CH₂C₆H₅ or OCOC(CH₃)₃,
R¹ denotes H, or a linear or branched alkyl group having 1 to 30 C atoms, optionally substituted by OH, OR, SH, SR, NH₂, NHR, NR₂, substituted or unsubstituted guanidinium, (NR₃)⁺, COOH, COOR, CONH₂, CONHR, CONR₂, COR, Ar or Het,
R in each case, independently of one another, optionally substituted, denotes a linear or branched alkyl group having 1 to 12 C atoms, cycloalkyl having 3 to 9 C atoms or cycloalkenyl having 5 to 9 C atoms,
Y denotes —CH₂— or —NZ—,
A and B each, independently of one another, denote H, R or OH, or A and B, together with the C atom to which they are linked, may form a carbonyl group,
n is 1, 2 or 3,
Ar denotes phenyl which is unsubstituted or mono-, di- or trisubstituted by R, OH or OR,
Het denotes a saturated or unsaturated mono- or bicyclic heterocyclic radical having 5 to 13 ring members, where 1, 2 or 3 N and/or 1 or 2 S or O atoms may be present, and the heterocyclic radical may be mono- or polysubstituted by R, OH or OR.
